(19) United States
(12) Patent Application Publication
(10) Pub. No.: US 2008/0300523 A1
(43) Pub. Date: Dec. 4, 2008

(54) THERAPEUTIC KNEE BRACE HAVING PRESSURE APPLICATORS

(76) Inventor: Dov Frishberg, New York, NY (US)

Correspondence Address: GREENBERG TRAURIG, LLP, 200 PARK AVE., P.O. BOX 677, FLORHAM PARK, NJ, 07932, US

(21) Appl. No.: 11/755,241

(22) Filed: May 30, 2007

(51) Int. Cl. A61F 5/00

(52) U.S. Cl. 602/26

(57) ABSTRACT

A brace for a knee joint including an elongated member and pressure applicators disposed thereon. Each of the pressure applicators may be inserted into a corresponding, closeable pouch on an inner surface of the elongated member. The pouches are arranged on the inner surface to situate the respective pressure applicators in the spaces between the various ligaments, cartilage and bones of the knee joint, so as to prevent applying pressure directly to these tissues and the pain caused thereby. A user may choose to insert all, or some, of the pressure applicators in their respective pouches, depending on nature of the injury. A method using the knee brace to facilitate healing of a knee joint injury is also disclosed.

### FIELD OF THE INVENTION

The present invention relates to therapeutic knee braces and, more particularly, to therapeutic knee brace having pressure applicators.

### BACKGROUND OF THE INVENTION

Reference is made to FIGS. 1A and 1B, which are anatomical representations of a right human knee joint KJ in extension and in flexion, respectively. The femur FR and tibia T bones of the leg meet to form the knee joint KJ. The fibula FB extends along the lateral side of the tibia T and joins the tibia T at the top, or head, thereof. The patella P (i.e., kneecap) is disposed proximate the femur FR and protects the front of the knee joint KJ. Cartilage C covers the articulating surfaces of the patella P, femur FR and tibia T, and acts to cushion these bones. The meniscus M is a layer of cartilage disposed between the femur FR and the tibia T and further cushions these bones.

With continued reference to FIGS. 1A and 1B, the ligaments of the knee joint KJ connect the associated bones to stabilize the joint. More particularly, the medial collateral ligament MCL connects the femur FR and the tibia T on the medial side of the knee joint KJ, and the lateral collateral ligament LCL connects the femur FR to the fibula FB on the lateral side of the knee joint KJ. The medial and lateral collateral ligaments MCL, LCL limit sideways motion of the knee joint KJ. The anterior cruciate ligament ACL connects the femur FR to the tibia T at the center of the knee joint KJ (see FIG. 1B), and limits rotation and forward rotation of the tibia T. The posterior cruciate ligament PCL also connects the femur FR arid the tibia T at the center of the knee (see FIG. 1B), and limits backward rotation of the tibia T.

Soft tissue injuries frequently occur within the knee joint KJ, including sprains and tears of the various ligaments discussed in the preceding paragraph. As a result, the knee joint KJ may become destabilised and may therefore require a brace to be worn for support, even after surgical correction of the injury. Such braces may be worn exclusively for athletic activity. Alternatively, knee braces may be worn throughout the day for ongoing stability, especially in cases where the injury is severe.

Several types of knee braces are available, ranging from prefabricated, lightweight athletic braces to custom-made braces incorporating support structures (e.g., splints and hinges). Many of these braces suffer from design shortcomings which exacerbate the knee injuries that lead users to wear such braces in the first place, thereby compromising any therapeutic value they may have. For instance, some available braces exert pressure, directly or indirectly, on the ligament(s) of the knee joint KJ, further stressing and/or straining such ligaments. Other braces restrict blood flow to the knee joint KJ when worn. These design shortcomings are especially problematic when the injured ligament(s), or other soft tissue (e.g., cartilage) is adversely affected.

### SUMMARY OF THE INVENTION

The knee brace of the present invention includes a body having inner and outer surfaces and which is sized and shaped so as to be applied to the knee. A plurality of pressure applicators are disposed on the inner surface of the body, and are arranged such that they substantially nestle an associated patella of the knee when the body is applied to the knee. A fastener member is connected to the body, and secures the body to the knee such that the applicators apply pressure against a joint of the knee.

The present invention also provides a method for facilitating healing of a knee joint injury using the brace. The method includes the step of securing the body against the knee such that the applicators substantially nestle an associated patella of the knee and such that at least one of the applicators applies pressure against a joint in the knee.

### BRIEF DESCRIPTION OF THE DRAWINGS

For a better understanding of the present invention, reference is made to the following detailed description of exemplary embodiments considered in conjunction with the accompanying drawings, in which:

FIG. 1A is an anatomical representation of a right knee joint area with the knee joint in extension, viewed from the front;

FIG. 1B is a view similar to FIG. 1A, except that the right knee joint is in flexion;

FIG. 2 is a front perspective view of a knee brace constructed in accordance with an exemplary embodiment of the present invention;

FIG. 3 is a rear perspective view of the knee brace shown in FIG. 2;

FIG. 4 is a front view of the knee brace of FIGS. 2 and 3 applied to a user's knee;

FIG. 5 is a rear perspective view of a knee brace of FIG. 2 which has been modified to include additional elements; and

FIG. 6 is a rear view of the knee brace of FIG. 5 applied to a user's knee.

### DETAILED DESCRIPTION OF THE INVENTION

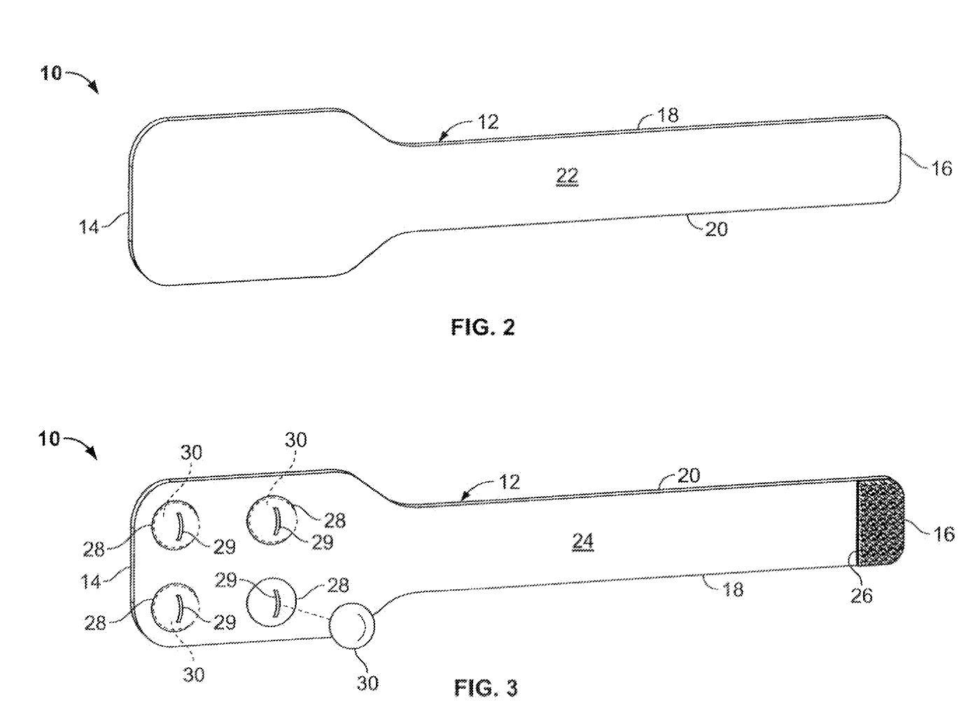

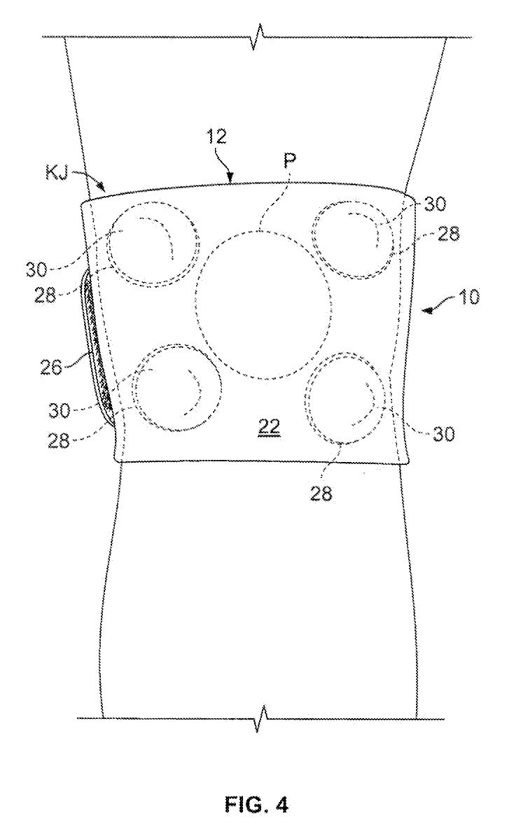

FIGS. 2-4 illustrate a knee brace 10 constructed in accordance with an exemplary embodiment of the present invention. The knee brace 10 includes a body, shown as an elongated member 12 having a pair of ends 14, 16, a pair of sides 18, 20, an outer surface 22 (see FIG. 2) and an inner surface 24 (see FIG. 3). The elongated member 12 has a similar construction to that of conventional athletic knee braces (e.g., such as those marketed by Becton Dickinson and Company, Franklin Lakes, N.J. under the trademark Tru-Fit®), and is preferably made of a breathable, lightweight, flexible and elastic material (or materials), such as those used in making conventional braces. As shown in FIGS. 2 and 3, the end 14 is preferably wider than the end 16. This shape facilitates the wrapping of the brace 10 about a user's knee joint KJ, as shown in FIG. 4. A fastener 26 is affixed to the inner surface 24 adjacent the end 16 (see FIG. 3) such that the end 16 can be secured to the outer surface 22 adjacent the end 14 when the brace 10 is placed on a user's knee (i.e., the brace 10 can be retained in place after it is wrapped about the knee joint KJ). The fastener 26 can be any type of conventional fastener. For instance, the fastener 26 can be a hook-type fastener, and the outer surface 22 can be constructed so as to function as a mating loop-type fastener. In this manner, the fastener 26 can be attached directly, but removably, to the outer surface 22.

Referring primarily to FIG. 3, pouches 28 are disposed on (e.g., sewn to) the inner surface 24 of the elongated member 12 proximate the end 14. Each of the pouches 28 is provided with a slit 29 and is made preferably from a flexible material. A pressure applicator 30 is removably placed in each of the pouches 28 through a corresponding one of the slits 29. While the pressure applicators 30 can have any desired shape, a spherical shape is particularly suitable for use in conjunction with the present invention. The pressure applicators 30 can also be made from any substantially rigid materials, such as metal, polyvinylchloride, etc. The pouches 28 are located such that the pressure applicators 30 can surround a user's patella P when the brace 10 is applied to the user's knee. In this regard, the pouches 28 can be arranged in any desirable formation. For instance, the pouches 28 can be arranged in an approximate square formation, as shown in FIG. 3.

The brace 10 is worn by a user about his/her injured knee joint KJ, as illustrated in FIG. 4. More particularly, when putting on the brace 10, the user places the inner surface 24 of the end 14 of the elongated member 12 against the knee joint KJ so that the pressure applicators 30 are arranged in the aforementioned approximate square formation in the lateral-medial plane of the knee joint KJ. In this arrangement, the pressure applicators 30 are arranged to surround, and stabilize or “nestle” the patella P. Once the pressure applicators 30 are placed about the patella P, the user wraps the elongated member 12 around the knee joint KJ and attaches the end 16 to the end 14 using the fastener 26, thereby securing the brace 10 and the pressure applicators 30 in place.

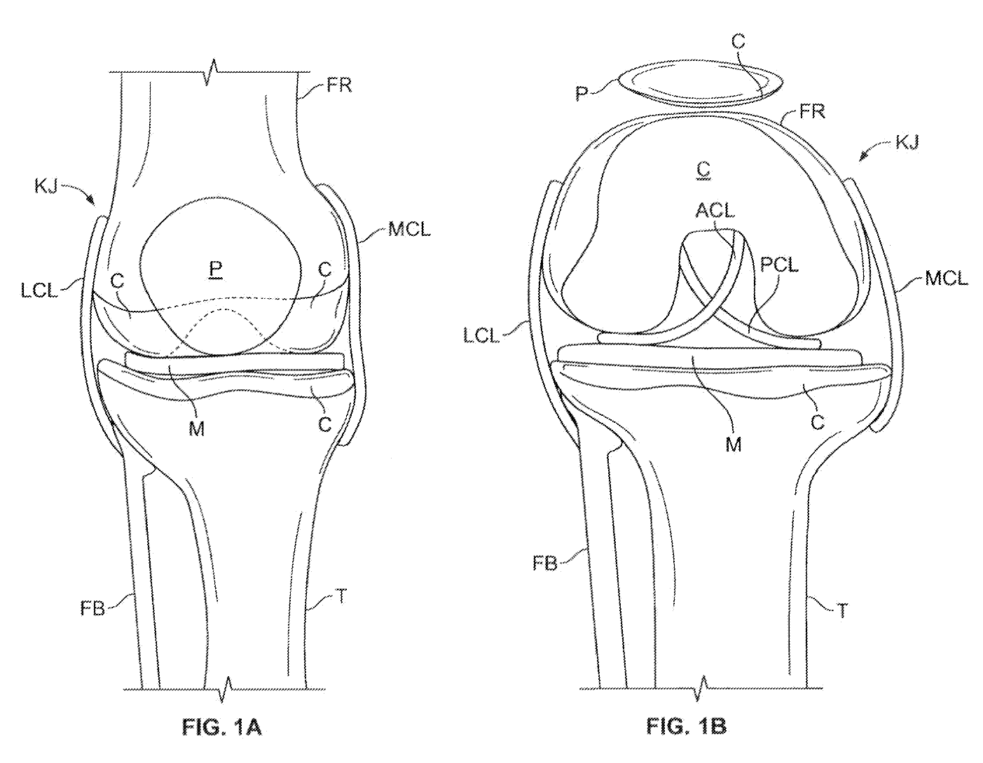

Without limiting the scope of the present invention, it is believed that when the pressure applicators 30 are arranged to surround or nestle the patella P, they tend to apply pressure in between the various ligaments, cartilage and/or bones of the knee joint KJ (see FIGS. 1A and 1B). For instance, the pressure applicators 30 tend to wedge themselves, at least partially, into spaces formed between the knee bones (e.g., the joint between the tibia T and the femur FR), thereby reducing or eliminating the pressure, and hence the pain, resulting from the bones pressing against each other when the cushioning cartridge is damaged and/or partially missing due to wear. In this regard, the pressure applicators 30 are designed so as not to stretch or strain damaged ligaments or to restrict blood flow thereto.

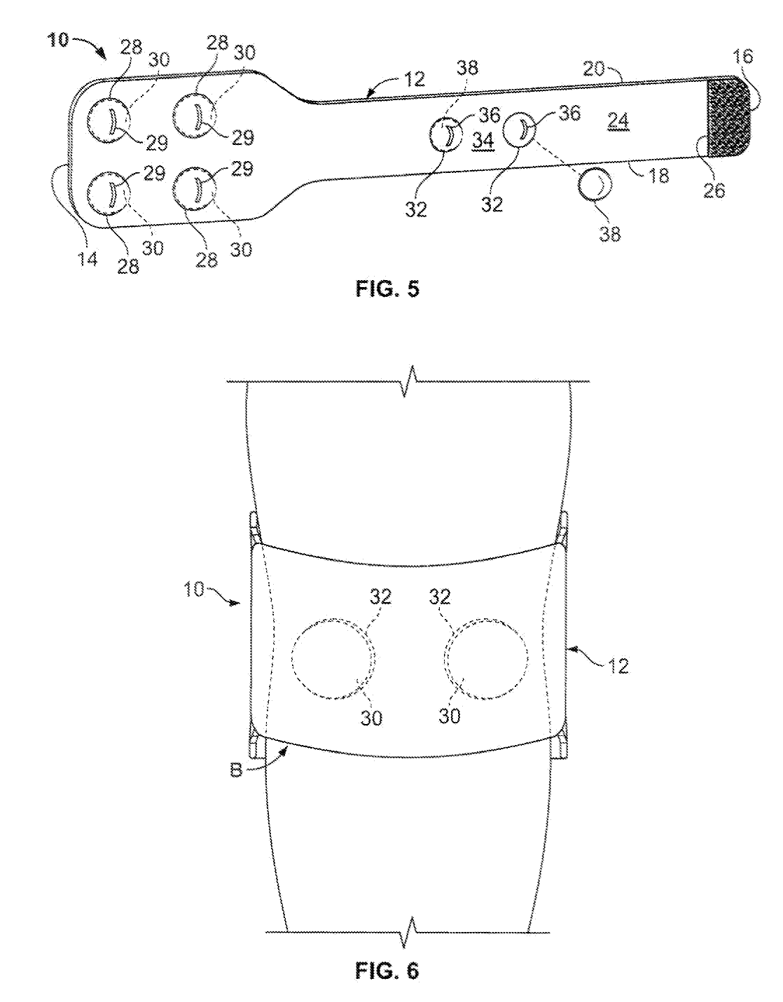

It should be noted that the present invention can have numerous modifications and variations. For instance, referring to FIGS. 5 and 6, the brace 10 can include one or more back pouches 32 disposed along a longitudinal axis on the inner surface 24 of the elongated member 12, proximate a location 34 intermediate the ends 14, 16. Each of the back pouches 32 has a slit 36 so as to removably receive a pressure applicator 38, which is similar, in construction and function, to the pressure applicators 30. When the user puts the brace 10 on his or her knee, the back pouches 32, and hence the pressure applicators 38, are positioned at the back B of the knee joint KJ, as illustrated in FIG. 6. Like the pressure applicators 30, the pressure applicators 38 are adapted to apply pressure between the bones, ligaments and cartridge of the knee joint KJ.

Further modifications may be made to the knee brace 10 of the present invention by the user. More particularly, rather than inserting all six of the pressure applicators 30, 38 in the respective pouches 28 and back pouches 32, a user may choose to insert fewer pressure applicators 30, 38 only in the pouches 28, 32 at locations corresponding to his or her injury. For example, if a user is experiencing pain resulting from cartilage damage in his knee joint, he may choose to only insert two pressure applicators 30 into respective pouches 28 on the side 18 of the elongated member 12 at locations selected to achieve the aforementioned “wedging” between the opposed ends of the tibia T and femur FR, and ultimately alleviate this pain. The user may remove the remaining two pressure applicators 30 from their corresponding pouches 28 on the side 20 of the elongated member 12, and not insert the two pressure applicators 38 in the respective back pouches 32.

Besides the modifications discussed above, additional modifications can be implemented in the brace 10. For instance, the number and spatial arrangement of the pressure applicators 30, 38 can be modified. The shape, size and/or construction of the elongated member 12 may also be modified. Further, the fastening member 26 can be replaced with any conventional fastening mechanisms.

An alternate embodiment of the knee brace of the present invention includes an elongated member having a different shape. For instance, rather than having a tapered, or “tongue” formation, wherein one end of the elongated member is wider then the other end (see FIGS. 2, 3 and 5), the elongated member may instead have a uniform width, wherein both ends have substantially equal widths. In this embodiment, the end distal to the pressure applicators would be wide enough to overlay the pressure applicators when the user secures the knee brace on his or her knee joint.

In yet another alternate embodiment of the present invention, the knee brace includes an endless sleeve or tubular member rather than an elongated member. The endless sleeve member enables the user to slide the knee brace on and off his or her knee joint rather than wrapping it around his or her knee joint, as is necessary with the elongated member.

It should be understood that the embodiments described herein are merely exemplary and that a person skilled in the art may make many variations and modifications thereto without departing from the spirit and scope of the present invention. All such variations and modifications, including those discussed above, are intended to be included within the scope of the invention as defined in the appended claims.

I claim:

1. A brace for a knee, comprising a body having inner and outer surfaces and sized and shaped so as to be applied to the knee; a plurality of pressure applicators disposed on said inner surface of said body, said applicators being arranged such that they substantially nestle an associated patella of the knee when said body is applied to the knee; and a fastener member connected to said body for securing said body to the knee such that said applicators apply pressure against a joint of the knee.

2. The brace of claim 1, wherein said applicators are positioned on said inner surface of said body in a rectangular pattern.

3. The brace of claim 2, further comprising a plurality of pouches positioned on said inner surface of said body, each of said pressure applicators being positioned in a corresponding one of said pouches.

4. The brace of claim 3, wherein each of said pouches includes a slit so as to allow a corresponding one of said pressure applicators to be removed therefrom.

5. The brace of claim 4, wherein each of said applicators has a spherical shape.

6. The brace of claim 5, further comprising at least one second pressure applicator positioned on said inner surface of said body so as to urge against a rear of the knee when said body is applied to the knee.

7. The brace of claim 6, further comprising at least one second pouch for removably receiving said at least one second pressure applicator.

8. The brace of claim 1, further comprising at least one second pressure applicator positioned on said Inner surface of said body so as to urge against a rear of the knee when said body is applied to the knee.

9. The brace of claim 8, wherein said body has a first section and a second section projecting from said first section, each of said plurality of pressure applicators being positioned on said first section, said at least one second pressure applicator being on said second section.

10. The brace of claim 9, wherein said fastening member is attached to said second section of said body such that said fastening member can be secured to said outer surface of said body adjacent said first section when said body is wrapped around the knee.

11. A method for facilitating healing of a knee joint injury using a brace including a body, which has inner and outer surfaces, and a plurality of pressure applicators, which are disposed on the inner surface of the body, said method comprising the step of securing the body against the knee such that the applicators substantially nestle an associated patella of the knee and such that at least one of the applicators applies pressure against a joint in the knee.

12. The method of claim 11, wherein the applicators are positioned on the inner surface of the body in a rectangular pattern.

13. The method of claim 12, wherein the brace further includes a plurality of pouches positioned on the inner surface of the body, each of the pressure applicators being positioned in a corresponding one of the pouches.

14. The method of claim 13, wherein each of the pouches includes a slit so as to allow a corresponding one of the pressure applicators to be removed therefrom.

15. The method of claim 14, wherein each of tire applicators has a spherical shape.

16. The method of claim 15, wherein the brace includes at least one pouch, which is positioned on the inner surface of the body, and at least one second pressure applicator, which is received in the at least one pouch so as to urge against a rear of the knee.

17. The method of claim 11, wherein the brace includes at least one second pressure applicator positioned on the inner surface of the body so as to urge against a rear of the knee when the body is applied to the knee.

18. The method of claim 17, wherein the body has a first section and a second section projecting from the first section, each of the plurality of pressure applicators being positioned on the first section, the at least one second pressure applicator being on the second section.

19. The method of claim 18, wherein the brace includes a fastening member attached to the second section of the body such that the fastening member can be secured to the outer surface of the body adjacent the first section when the body is wrapped around the knee.

20. The method of claim 11, wherein said securing step includes the steps of applying the body against the knee; wrapping the body around the knee; and affixing an end of the body to a surface of the body so as to fasten the brace to the knee.
